(19) United States
(12) Patent Application Publication
(10) Pub. No.: US 2015/0361131 A1
(43) Pub. Date: Dec. 17, 2015

(54) HIGH TEMPERATURE DEAD END ANTIBODY FILTRATION

(71) Applicant: NOVO NORDISK A/S, Bagsværd (DK)

(72) Inventor: Ole E. Jensen, Vanloese (DK)

(21) Appl. No.: 14/655,401

(22) PCT Filed: Dec. 30, 2013
(86) PCT No.: PCT/EP2013/078119

§ 371 Date: Jun. 25, 2015

Related U.S. Application Data
(60) Provisional application No. 61/748,295, filed on Jan. 2, 2013.

(30) Foreign Application Priority Data
Dec. 28, 2012 (EP) 12199578.1

(51) Int. Cl. C07K 1/34; C07K 16/38; C07K 16/24

(52) U.S. Cl. CPC C07K 1/34; C07K 16/244; C07K 16/38

(57) ABSTRACT

The invention relates to a method of dead end filtration of a highly concentrated antibody containing solutions at a temperature above room temperature, wherein the filter flow rate is increased compared to filtration at a room temperature, the filter flow rate is kept constant and the yield increased compared to filtration at a room temperature.

### TECHNICAL FIELD

The current invention relates to concentrated protein containing solutions, in particularly concentrated antibody containing solutions, particular filtration of such concentrated antibody containing solutions.

### BACKGROUND

A number of injectable protein containing solutions, particular antibody containing solutions are supplied to the market. The use of those solutions for subcutaneous injection, results in the demand of high concentration of the protein in the solutions due to limitations in injection volume.

Especially for highly concentrated protein solutions the sterile/germ filtration by pumping the solution through a filter, such as 0.22μ filter may be tedious or impossible and can result in very low yields and extremely low filter capacities because of the high viscosity of the solution. It also often results in a drop in protein content in filtrate and low filter capacity. The result is that applying high pressure may be necessary to maintain flow. This high pressure applies stress to the protein and may result in higher rate of impurities.

Most proteins are not stable at high temperatures and therefore likely to denature and loose activity at elevated temperature above +30° C. This has limited the use of raising the temperature of the highly concentrated protein solutions during filtration for lowering the viscosity. However, unexpectedly antibodies seem to be quite stable at higher temperature, as far as up to 50-70° C., making it feasible to filtrate them at higher temperature.

### SUMMARY

The current invention provides a method of filtration of a highly concentrated antibody and/or fragments solution. It furthermore provides a method of filtration of a highly concentrated solution comprising antibody or/and a fragment thereof in concentration above 100 g/L, wherein the solution is heated to a temperature above 30° C. during filtration. The current invention furthermore provides a method of filtration of a highly concentrated antibody solution, wherein the antibody or a fragment thereof, is present in concentration above 200 g/L, wherein the solution is heated to a temperature above 30° C. during filtration. The present invention furthermore provides a method of increasing the filtration yield during filtration of a highly concentrated antibody solution. The current invention furthermore provides a method of keeping the filter flow rate constant during filtration of a highly concentrated antibody solution. Furthermore the current invention provides a method of increasing the filter flow rate during the filtration of a highly concentrated antibody solution.

### DESCRIPTION

As explained above, filtration at room temperature of highly concentrated protein solutions is slow and may result in low recoveries or yields of the protein and may furthermore show a drop in the protein content in filtrate, while demonstrating low filter capacity. One traditional solution is to apply high pressure during the filtration to maintain flow. This use of high pressure applies stress to the protein which may result in denaturation of the protein and increase of other product related impurities.

It has surprisingly been discovered antibodies are more stable at higher temperature (above 30° C.) than expected and it is therefore suitable to perform filtration of highly concentrated antibody solutions at a higher temperature than expected. This filtration of a highly concentrated antibody solution at an elevated temperature significantly increase filtration speed and filter capacity without denaturation of the protein. Furthermore the protein recovery or yield is increased and protein content in filtrate is maintained.

As demonstrated an inline heat exchanger situated just before the filter to heat up the solution pumped through the filter reduced the filtration time markedly and increased yield for the filtration or the heating of the solution could be done in other ways i.e. by placing the filter in an oven/water bath at the appropriate temperature.

The term “protein”, “polypeptide” and “peptide” as used herein means a compound composed of at least five constituent amino acids connected by peptide bonds. The constituent amino acids may be from the group of the amino acids encoded by the genetic code and they may be natural amino acids which are not encoded by the genetic code, as well as synthetic amino acids. Natural amino acids which are not encoded by the genetic code are e.g. hydroxyproline, y-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (a-aminoisobutyric acid), Abu (α-aminobutyric acid), Tle (tert-butylglycine), β-alanine, 3-aminomethyl benzoic acid and anthranilic acid.

The term “antibody” covers monoclonal antibodies (including full length antibodies which have an immunoglobulin Fc region), antibody compositions with polyepitopic specificity, bispecific antibodies, diabodies, and single-chain molecules, as well as antibody fragments (e. g., Fab, F(ab′)₂, and Fv).

The term “monoclonal antibody” as used herein refers to an antibody obtained from a population of substantially homogeneous antibodies, i. e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256: 495 (1975), or may be made by recombinant DNA methods (see, e. g., U.S. Pat. No. 4,816,567). The “monoclonal antibodies” may also be isolated from phage antibody libraries using the techniques described in Clackson et al., Nature, 352: 624-628 (1991) and Marks et al., J. Mol. Biol., 222: 581-597 (1991), for example.

The monoclonal antibodies herein may extend to include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain (s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)).

Examples of suitable antibodies, which may be formulated in a stable composition of the invention include: 3F8, Abagovomab, Abciximab, ACZ885 (canakinumab), Adalimumab, Adecatumumab, Afelimomab, Afutuzumab, Alacizumab pegol, Alemtuzumab, Altumomab pentetate, Anatumomab mafenatox, Anrukinzumab (IMA-638), Apolizumab, Arcitumomab, Aselizumab, Atlizumab (tocilizumab), Atorolimumab, Bapineuzumab, Basiliximab, Bavituximab, Bectumomab, Belimumab, Bertilimumab, Besilesomab, Bevacizumab, Biciromab, Bivatuzumab mertansine, Blinatumomab, Brentuximab vedotin, Briakinumab, Canakinumab, Cantuzumab mertansine, Capromab pendetide, Catumaxomab, Cedelizumab, Certolizumab pegol, Cetuximab, Citatuzumab bogatox, Cixutumumab, Clenoliximab, Clivatuzumab tetraxetan, CNTO 148 (golimumab), CNTO 1275 (ustekinumab), Conatumumab, Dacetuzumab, Daclizumab, Denosumab, Detumomab, Dorlimomab aritox, Dorlixizumab, Ecromeximab, Eculizumab, Edobacomab, Edrecolomab, Efalizumab, Efungumab, Elsilimomab, Enlimomab pegol, Epitumomab cituxetan, Epratuzumab, Erlizumab, Ertumaxomab, Etaracizumab, Exbivirumab, Fanolesomab, Faralimomab, Felvizumab, Fezakinumab, Figitumumab, Fontolizumab, Foravirumab, Fresolimumab, Galiximab, Gantenerumab, Gavilimomab, Gemtuzumab ozogamicin, Golimumab, Gomiliximab, Ibalizumab, Ibritumomab tiuxetan, Igovomab, lmciromab, Infliximab, Intetumumab, Inolimomab, Inotuzumab ozogamicin, Ipilimumab, Iratumumab, Keliximab, Labetuzumab, Lebrikizumab, Lemalesomab, Lerdelimumab, Lexatumumab, Libivirumab, Lintuzumab, Lucatumumab, Lumiliximab, Mapatumumab, Maslimomab, Matuzumab, Mepolizumab, Metelimumab, Milatuzumab, Minretumomab, Mitumomab, Morolimumab, Motavizumab, Muromonab-CD3, MYO-029 (stamulumab), Nacolomab tafenatox, Naptumomab estafenatox, Natalizumab, Nebacumab, Necitumumab, Nerelimomab, Nimotuzumab, Nofetumomab merpentan, Ocrelizumab, Odulimomab, Ofatumumab, Omalizumab, Oportuzumab monatox, Oregovomab, Otelixizumab, Pagibaximab, Palivizumab, Panitumumab, Panobacumab, Pascolizumab, Pemtumomab, Pertuzumab, Pexelizumab, Pintumomab, Priliximab, Pritumumab, PRO 140, Rafivirumab, Ramucirumab, Ranibizumab, Raxibacumab, Regavirumab, Reslizumab, Rilotumumab, Rituximab, Robatumumab, Rontalizumab, Rovelizumab, Ruplizumab, Satumomab, Sevirumab, Sibrotuzumab, Sifalimumab, Siltuximab, Siplizumab, Solanezumab, Sonepcizumab, Sontuzumab, Stamulumab, Sulesomab, Tacatuzumab tetraxetan, Tadocizumab, Talizumab, Tanezumab, Taplitumomab paptox, Tefibazumab, Telimomab aritox, Tenatumomab, Teneliximab, Teplizumab, TGN1412, Ticilimumab (tremelimumab), Tigatuzumab, TNX-355 (ibalizumab), TNX-650, TNX-901 (talizumab), Tocilizumab (atlizumab), Toralizumab, Tositumomab, Trastuzumab, Tremelimumab, Tucotuzumab celmoleukin, Tuvirumab, Urtoxazumab, Ustekinumab, Vapaliximab, Vedolizumab, Veltuzumab, Vepalimomab, Visilizumab, Volociximab, Votumumab, Zalutumumab, Zanolimumab, Ziralimumab, Zolimomab aritox and the like.

In one embodiment, the protein is an immunoglobulin. In one embodiment, the protein is an antibody. In one embodiment, the protein is a monoclonal antibody (mAb). In one embodiment, the protein is an IgG4 antibody.

In one embodiment, the antibody is a monoclonal anti-IL20 antibody. In one embodiment, the antibody is an anti-IL20 antibody as described in WO2010/000721. In one embodiment, the anti-IL20 monoclonal antibody is 15D2 or 5B7 as described in WO2010/000721.

In one embodiment, the antibody is a monoclonal anti-TFPI monoclonal antibody. In one embodiment, the antibody is an anti-TFPI antibody as described in PCT2009EP067598. In one embodiment, the anti-TFPI monoclonal antibody is HzTFPI4F36 as described in PCT2009EP067598.

It will be appreciated that the invention finds particular utility where the protein/antibody is present in solution to be filtrated in high concentrations. Thus, in one embodiment, the protein is present in a concentration of 50 mg/ml or more, such as 55, 60, 65, 70, 75, 80, 85, 90, 95, 100, 150, 200, 201, 202, 203, 204, 205, 206, 207, 208, 209, 210, 215, 220, 222, 224, 226, 228, 230, 235, 240, 245, 250, 300, 350 mg/ml or more. In one embodiment, the protein is present within the composition in an amount of between 50 mg/ml and 300 mg/ml, for instance between 50 mg/ml and 250 mg/ml, such as between 50 mg/ml and 200 mg/ml, for instance between 50 mg/ml and 150 mg/ml. In one embodiment, the protein is present in a concentration of between 75 mg/ml and 350 mg/ml, such as between 75 mg/ml and 300 mg/ml, for instance between 75 mg/ml and 250 mg/ml, such as between 75 mg/ml and 200 mg/ml, for instance between 75 mg/ml and 150 mg/ml. In one embodiment, the protein is present in a concentration of between 100 mg/ml and 350 mg/ml, such as between 100 mg/ml and 300 mg/ml, for instance between 100 mg/ml and 250 mg/ml, such as between 100 mg/ml and 200 mg/ml, for instance between 100 mg/ml and 150 mg/ml, such as between 150 mg/ml and 300 mg/ml, for instance between 150 mg/ml and 250 mg/ml, such as between 150 mg/ml and 200 mg/ml, for instance between 200 mg/ml and 300 mg/ml, such as between 200 mg/ml and 250 mg/ml or for instance between 250 mg/ml and 300 mgl/ml.

The term “stability” of a protein in a composition as used herein refers to the biological stability, physical stability or chemical stability of the protein in solution. Chemical covalent changes in the protein structure leading to formation of chemical degradation products with potential less biological potency and/or potential increased immunogenic properties compared to the native protein structure, during manufacturing process. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein composition is well-known by the person skilled in the art. Most proteins are prone to deamidation, a process in which the side chain amide group in glutaminyl or asparaginyl residues is hydrolysed to form a free carboxylic acid. Other degradation pathways involve formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein composition can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

There are various analytical techniques for measuring protein stability available in the art (Peptide and Protein Drug Delivery, 247-301, Vincent Lee Ed. & Marcel Dekker, NY. Pubs 1991; and Jones, A. Adv. Drug Delivery Rev. 10: 29-90, 1993).

SEC-HPLC is in particular used for quantification of protein aggregates. The samples may for instance be analysed using a TSK G3000 SWXL column, isocratic elution and subsequent UV detection at 214 or 280 nm. This method is used to determine monomeric IgG content and % High Molecular Weight Proteins (HMWP) consisting of dimeric species or larger which are separated according to size by the gel resin. The monomeric content and % HMWP are determined relative to the total protein content detected by the method.

Physical stability of protein solution can be measured by well-known methods, including measurement of attenuation of light by measurement of absorbance or optical density. Such measurements relate to the turbidity of solution.

Denature (of protein) as referred to in this application is a process in which proteins or nucleic acids lose the tertiary structure and secondary structure which is present in their native state, by application of some external stress or compound such as a strong acid or base, a concentrated inorganic salt, an organic solvent (e.g., alcohol or chloroform), or heat. If proteins in a living cell are denatured, this results in disruption of cell activity and possibly cell death. Denatured proteins can exhibit a wide range of characteristics, from loss of solubility to communal aggregation.

Viscosity as used herein is used as the absolute viscosity also termed dynamic viscosity. Measurements are done by the cone and plate technique with a Peltier element set at 25° C., and where a well-defined shear stress gradient is applied to a sample and the resulting shear rate is measured. The viscosity is the ratio of the shear stress to the shear rate. Absolute viscosity is expressed in units of centipoise (cP) at 25° C.

The term filtration as used herein refers to a dead-end filtration. A dead-end (normal/direct/regular) filtration is the mechanical or physical operation which is used for the separation of solids from fluids (liquids or gases) by interposing a medium, through which only the fluid can pass. Oversize solids in the fluid are retained, but the separation is not complete; solids will be contaminated with some fluid and filtrate will contain fine particles (depending on the pore size and filter thickness). In the dead-end filtration technique the filtration medium is a membrane and all the fluid passes through the membrane, and all particles larger than the pore size of the membrane are retained on its surface. Trapped particles will start to build up a “filter cake” on the surface of the membrane, which has an impact on the efficiency of the filtration process.

Microfiltration is widely used in dead end filtration and is a way of removing particles in the size range of 0.1 to 10 μm from fluids, by passing the fluid through a microporous medium or membrane. The term microfiltration, as used herein, refers to a a such dead end filtration for removal of particles in the 0.1 to 10 μm size range, and not to tangential flow micro filtration.

The term filtrate, as used herein, refers to the fluid of the gas that has passed through the membrane and has been filtered.

The term “room temperature” as used herein, refers to a temperature between 18 and 25° C.

The term filter flow rate as used herein, refer to the speed of the flow through the filter or rate the filtrate passes the filter.

### EXAMPLES

In those examples the concentrated antibody (mAb) solution is heated to the desired temperature by a heat exchanger situated in-line just before the filter or alternatively the mAb solution is heated by placing the solution, filter(s) and other equipment in a thermostated oven before filtering.

### Example 1

The example was carried out to investigate differences in 0.22 μm filtering of a high concentrated mAb solution at room temperature (20° C.) and at 40° C.

A solution of anti TFPI, 204 g/l, 10 mM Histidine, pH 6.0, was pumped by a peristaltic pump through a Sartobran 150 filter cartridge (0.45+0.22 micron combi filter) at 20° C. The resulting flow was low (1.5 mL/min) and decreased to zero after filtering 100 mL. The filtrate mAb concentration was 186 g/L.

A heat exchanger (Exergy, serie 17 model 00402) was mounted in-line before the filter cartridge and a 150 mL anti TFPI solution, 204 g/l, 10 mM Histidine, pH 6.0, was pumped by a peristaltic pump through a fresh Sartobran 150 filter cartridge (0.45+0.22 micron combi filter) at 40° C. The resulting flow was constantly 50 mL/min. and the filtrate concentration was 200 g/L. The level of high molecular weight protein (HMWP) as well as deamidated forms were unaffected by the filtration at either temperature. The mAb concentrations were determined by NanoDrop 2000C instrument, 280 nm, (Thermo Scientific) and samples were pre-diluted 10 times with 0.9% NaCl.

The conclusion is that the filtration at elevated temperature is both much faster (higher flux) and the yield is much better, while surprisingly the formation of deamidated forms or denatured antibodies, as measured by %HMWP did not change.

### Example 2

The following 3 examples were carried out to investigate the effect of 0.22 μm filtration on product concentration and at a new mAb and by placing the complete filtration system in a thermostated oven.

A solution of:

226 mg/ml Anti-IL-20

150 mM Sucrose

25 mM Arg

25 mM NaCl

33 mM His

pH 6.5

Was filled into 20 mL syringe which was placed in an oven thermostated at 45° C. together with filters (33 mm MillexGV, 0.22 μm and prefilter 33 mm Millex AA, 0.8 μm).

The solution was filtered through 0.8 μm prefilter and 0.22 μm filter connected i series without any problems by manually pressurising the syringe piston.

The antibody concentration was measured by a NanoDrop 2000C (Thermo Scientific) instrument, 280 nm, after dilution 10 times with 0.9% NaCl. The filtrate concentration was determined to be 219 mg/ml.

A solution of:

222 mg/ml Anti-IL-20

150 mM Sucrose

25 mM Arg

25 mM NaCl

33 mM His

pH 6.5

Was filled into a 10 mL syringe which was placed in an oven temperated at 45° C. together with filters (33 mm MillexGV, 0.22 μm and prefilter 33 mm Millex AA, 0.8 μm).

The solution was filtered through 0.8 μm prefilter and 0.22 μm filter connected i series without any problems by manually pressurising the syringe piston.

The antibody concentration was measured by a NanoDrop 2000C (Thermo Scientific) instrument, 280 nm, after dilution 10 times with 0.9% NaCl. The filtrate concentration was determined to be 219 mg/ml.

A solution of:

224 mg/ml Anti-IL-20

25 mM Arg

100 mM NaCl

66 mM His

pH 6.5

Was filled into a 10 mL syringe which was placed in an oven temperated at 45° C. together with filters (33 mm MillexGV, 0.2μ um and prefilter 33 mm Millex AA, 0.8 μm).

The solution was filtered through 0.8 μm prefilter and 0.22 μm filter connected i series without any problems by manually pressurising the syringe piston.

The antibody concentration was measured by a NanoDrop 2000C (Thermo Scientific) instrument, 280 nm, after dilution 10 times with 0.9% NaCl. The filtrate concentration was determined to be 226 mg/ml.

It may be concluded from the three different experiments that 0.22 μm filtrations may be carried out with different antibodies and completed in the way described without any significant loss in product concentration.

### EMBODIMENTS

The following is a non-limiting list of embodiments of the present invention.
1. A method of filtration of a highly concentrated solution containing an antibody and/or a fragment thereof.
2. A method of increasing the yield during the filtration of a highly concentrated solution containing an antibody and/or a fragment thereof, wherein the solution is heated to a temperature ranging from 30-70° C. during filtration.
3. A method according to embodiment 2, wherein the yield is more than 100% increased.
4. A method according to embodiment 2, wherein the yield is more than 200% increased.
5. A method according to embodiment 2, wherein the yield is more than 300% increased.
6. A method according to embodiment 2, wherein the yield is more than 400% increased.
7. A method according to embodiment 2, wherein the yield is more than 500% increased.
8. A method according to embodiment 2, wherein the yield is more than 600% increased.
9. A method according to embodiment 2, wherein the yield is more than 700% increased.
10. A method according to embodiment 2, wherein the yield is more than 800% increased.
11. A method according to embodiment 2, wherein the yield is more than 900% increased.
12. A method according to embodiment 2, wherein the yield is more than 1000% increased.
13. A method according to embodiment 2, wherein the yield is more than 1100% increased.
14. A method according to embodiment 2, wherein the yield is more than 1200% increased.
15. A method according to embodiment 2, wherein the yield is more than 1300% increased.
16. A method according to embodiment 2, wherein the yield is more than 1400% increased.
17. A method according to embodiment 2, wherein the yield is more than 1500% increased.
18. A method according to embodiment 2, wherein the yield is more than 1600% increased.
19. A method according to embodiment 2, wherein the yield is more than 1700% increased.
20. A method according to embodiment 2, wherein the yield is more than 1800% increased.
21. A method according to embodiment 2, wherein the yield is more than 1900% increased.
22. A method according to embodiment 2, wherein the yield is more than 2000% increased.
23. A method according to embodiment 2, wherein the yield is more than 2100% increased.
24. A method according to embodiment 2, wherein the yield is more than 2200% increased.
25. A method according to embodiment 2, wherein the yield is more than 2300% increased.
26. A method according to embodiment 2, wherein the yield is more than 2400% increased.
27. A method according to embodiment 2, wherein the yield is more than 2500% increased.
28. A method according to embodiment 2, wherein the yield is more than 2600% increased.
29. A method according to embodiment 2, wherein the yield is more than 2700% increased.
30. A method according to embodiment 2, wherein the yield is more than 2800% increased.
31. A method according to embodiment 2, wherein the yield is more than 2900% increased.
32. A method according to embodiment 2, wherein the yield is more than 3000% increased.
33. A method according to any of the preceding embodiments, wherein the concentrated antibody solution contains 50 mg/ml or more of antibody/antibody fragments.
34. A method according to any of the preceding embodiments, wherein the concentrated antibody solution contains 55 mg/ml or more of antibody/antibody fragments.
35. A method according to any of the preceding embodiments, wherein the concentrated antibody solution contains 60 mg/ml or more of antibody/antibody fragments.
36. A method according to any of the preceding embodiments, wherein the concentrated antibody solution contains 65 mg/ml or more of antibody/antibody fragments.
37. A method according to any of the preceding embodiments, wherein the concentrated antibody solution contains 70 mg/ml or more of antibody/antibody fragments.
38. A method according to any of the preceding embodiments, wherein the concentrated antibody solution contains 75 mg/ml or more of antibody/antibody fragments.
39. A method according to any of the preceding embodiments, wherein the concentrated antibody solution contains 80 mg/ml or more of antibody/antibody fragments.
40. A method according to any of the preceding embodiments, wherein the concentrated antibody solution contains 85 mg/ml or more of antibody/antibody fragments.
41. A method according to any of the preceding embodiments, wherein the concentrated antibody solution contains 90 mg/ml or more of antibody/antibody fragments.
42. A method according to any of the preceding embodiments, wherein the concentrated antibody solution contains 95 mg/ml or more of antibody/antibody fragments.
43. A method according to any of the preceding embodiments, wherein the concentrated antibody solution contains 100 mg/ml or more of antibody/antibody fragments.
44. A method according to any of the preceding embodiments, wherein the concentrated antibody solution contains 105 mg/ml or more of antibody/antibody fragments.
45. A method according to any of the preceding embodiments, wherein the concentrated antibody solution contains 110 mg/ml or more of antibody/antibody fragments.
46. A method according to any of the preceding embodiments, wherein the concentrated antibody solution contains 115 mg/ml or more of antibody/antibody fragments.
47. A method according to any of the preceding embodiments, wherein the concentrated antibody solution contains 120 mg/ml or more of antibody/antibody fragments.
48. A method according to any of the preceding embodiments, wherein the concentrated antibody solution contains 125 mg/ml or more of antibody/antibody fragments.
49. A method according to any of the preceding embodiments, wherein the concentrated antibody solution contains 130 mg/ml or more of antibody/antibody fragments.
50. A method according to any of the preceding embodiments, wherein the concentrated antibody solution contains 135 mg/ml or more of antibody/antibody fragments.
51. A method according to any of the preceding embodiments, wherein the concentrated antibody solution contains 140 mg/ml or more of antibody/antibody fragments.
52. A method according to any of the preceding embodiments, wherein the concentrated antibody solution contains 145 mg/ml or more of antibody/antibody fragments.
53. A method according to any of the preceding embodiments, wherein the concentrated antibody solution contains 150 mg/ml or more of antibody/antibody fragments.
54. A method according to any of the preceding embodiments, wherein the concentrated antibody solution contains 155 mg/ml or more of antibody/antibody fragments.
55. A method according to any of the preceding embodiments, wherein the concentrated antibody solution contains 160 mg/ml or more of antibody/antibody fragments.
56. A method according to any of the preceding embodiments, wherein the concentrated antibody solution contains 165 mg/ml or more of antibody/antibody fragments.
57. A method according to any of the preceding embodiments, wherein the concentrated antibody solution contains 170 mg/ml or more of antibody/antibody fragments.
58. A method according to any of the preceding embodiments, wherein the concentrated antibody solution contains 175 mg/ml or more of antibody/antibody fragments.
59. A method according to any of the preceding embodiments, wherein the concentrated antibody solution contains 180 mg/ml or more of antibody/antibody fragments.
60. A method according to any of the preceding embodiments, wherein the concentrated antibody solution contains 185 mg/ml or more of antibody/antibody fragments.
61. A method according to any of the preceding embodiments, wherein the concentrated antibody solution contains 190 mg/ml or more of antibody/antibody fragments.
62. A method according to any of the preceding embodiments, wherein the concentrated antibody solution contains 200 mg/ml or more of antibody/antibody fragments.
63. A method according to any of the preceding embodiments, wherein the concentrated antibody solution contains 201 mg/ml or more of antibody/antibody fragments.
64. A method according to any of the preceding embodiments, wherein the concentrated antibody solution contains 202 mg/ml or more of antibody/antibody fragments.
65. A method according to any of the preceding embodiments, wherein the concentrated antibody solution contains 203 mg/ml or more of antibody/antibody fragments.
66. A method according to any of the preceding embodiments, wherein the concentrated antibody solution contains 204 mg/ml or more of antibody/antibody fragments.
67. A method according to any of the preceding embodiments, wherein the concentrated antibody solution contains 205 mg/ml or more of antibody/antibody fragments.
68. A method according to any of the preceding embodiments, wherein the concentrated antibody solution contains 206 mg/ml or more of antibody/antibody fragments.
69. A method according to any of the preceding embodiments, wherein the concentrated antibody solution contains 207 mg/ml or more of antibody/antibody fragments.
70. A method according to any of the preceding embodiments, wherein the concentrated antibody solution contains 208 mg/ml or more of antibody/antibody fragments.
71. A method according to any of the preceding embodiments, wherein the concentrated antibody solution contains 209 mg/ml or more of antibody/antibody fragments.
72. A method according to any of the preceding embodiments, wherein the concentrated antibody solution contains 210 mg/ml or more of antibody/antibody fragments.
73. A method according to any of the preceding embodiments, wherein the concentrated antibody solution contains 211 mg/ml or more of antibody/antibody fragments.
74. A method according to any of the preceding embodiments, wherein the concentrated antibody solution contains 212 mg/ml or more of antibody/antibody fragments.
75. A method according to any of the preceding embodiments, wherein the concentrated antibody solution contains 213 mg/ml or more of antibody/antibody fragments.
76. A method according to any of the preceding embodiments, wherein the concentrated antibody solution contains 214 mg/ml or more of antibody/antibody fragments.
77. A method according to any of the preceding embodiments, wherein the concentrated antibody solution contains 215 mg/ml or more of antibody/antibody fragments.
78. A method according to any of the preceding embodiments, wherein the concentrated antibody solution contains 216 mg/ml or more of antibody/antibody fragments.
79. A method according to any of the preceding embodiments, wherein the concentrated antibody solution contains 217 mg/ml or more of antibody/antibody fragments.
80. A method according to any of the preceding embodiments, wherein the concentrated antibody solution contains 218 mg/ml or more of antibody/antibody fragments.
81. A method according to any of the preceding embodiments, wherein the concentrated antibody solution contains 219 mg/ml or more of antibody/antibody fragments.
82. A method according to any of the preceding embodiments, wherein the concentrated antibody solution contains 220 mg/ml or more of antibody/antibody fragments.
83. A method according to any of the preceding embodiments, wherein the concentrated antibody solution contains 221 mg/ml or more of antibody/antibody fragments.
84. A method according to any of the preceding embodiments, wherein the concentrated antibody solution contains 222 mg/ml or more of antibody/antibody fragments.
85. A method according to any of the preceding embodiments, wherein the concentrated antibody solution contains 223 mg/ml or more of antibody/antibody fragments.
86. A method according to any of the preceding embodiments, wherein the concentrated antibody solution contains 224 mg/ml or more of antibody/antibody fragments.
87. A method according to any of the preceding embodiments, wherein the concentrated antibody solution contains 224 mg/ml or more of antibody/antibody fragments.
88. A method according to any of the preceding embodiments, wherein the concentrated antibody solution contains 226 mg/ml or more of antibody/antibody fragments.
89. A method according to any of the preceding embodiments, wherein the concentrated antibody solution contains 227 mg/ml or more of antibody/antibody fragments.
90. A method according to any of the preceding embodiments, wherein the concentrated antibody solution contains 228 mg/ml or more of antibody/antibody fragments.
91. A method according to any of the preceding embodiments, wherein the concentrated antibody solution contains 229 mg/ml or more of antibody/antibody fragments.
92. A method according to any of the preceding embodiments, wherein the concentrated antibody solution contains 230 mg/ml or more of antibody/antibody fragments.
93. A method according to any of the preceding embodiments, wherein the concentrated antibody solution contains 231 mg/ml or more of antibody/antibody fragments.
94. A method according to any of the preceding embodiments, wherein the concentrated antibody solution contains 232 mg/ml or more of antibody/antibody fragments.
95. A method according to any of the preceding embodiments, wherein the concentrated antibody solution contains 233 mg/ml or more of antibody/antibody fragments.
96. A method according to any of the preceding embodiments, wherein the concentrated antibody solution contains 234 mg/ml or more of antibody/antibody fragments.
97. A method according to any of the preceding embodiments, wherein the concentrated antibody solution contains 235 mg/ml or more of antibody/antibody fragments.
98. A method according to any of the preceding embodiments, wherein the concentrated antibody solution contains 240 mg/ml or more of antibody/antibody fragments.
99. A method according to any of the preceding embodiments, wherein the concentrated antibody solution contains 245 mg/ml or more of antibody/antibody fragments.
100. A method according to any of the preceding embodiments, wherein the concentrated antibody solution contains 250 mg/ml or more of antibody/antibody fragments.
101. A method according to any of the preceding embodiments, wherein the concentrated antibody solution contains 255 mg/ml or more of antibody/antibody fragments.
102. A method according to any of the preceding embodiments, wherein the concentrated antibody solution contains 260 mg/ml or more of antibody/antibody fragments.
103. A method according to any of the preceding embodiments, wherein the concentrated antibody solution contains 265 mg/ml or more of antibody/antibody fragments.
104. A method according to any of the preceding embodiments, wherein the concentrated antibody solution contains 270 mg/ml or more of antibody/antibody fragments.
105. A method according to any of the preceding embodiments, wherein the concentrated antibody solution contains 275 mg/ml or more of antibody/antibody fragments.
106. A method according to any of the preceding embodiments, wherein the concentrated antibody solution contains 280 mg/ml or more of antibody/antibody fragments.
107. A method according to any of the preceding embodiments, wherein the concentrated antibody solution contains 285 mg/ml or more of antibody/antibody fragments.
108. A method according to any of the preceding embodiments, wherein the concentrated antibody solution contains 290 mg/ml or more of antibody/antibody fragments.
109. A method according to any of the preceding embodiments, wherein the concentrated antibody solution contains 295 mg/ml or more of antibody/antibody fragments.
110. A method according to any of the preceding embodiments, wherein the concentrated antibody solution contains 300 mg/ml or more of antibody/antibody fragments.
111. A method according to any of the preceding embodiments, wherein the concentrated antibody solution contains 305 mg/ml or more of antibody/antibody fragments.
112. A method according to any of the preceding embodiments, wherein the concentrated antibody solution contains 310 mg/ml or more of antibody/antibody fragments.
113. A method according to any of the preceding embodiments, wherein the concentrated antibody solution contains 315 mg/ml or more of antibody/antibody fragments.
114. A method according to any of the preceding embodiments, wherein the concentrated antibody solution contains 320 mg/ml or more of antibody/antibody fragments.
115. A method according to any of the preceding embodiments, wherein the concentrated antibody solution contains 325 mg/ml or more of antibody/antibody fragments.
116. A method according to any of the preceding embodiments, wherein the concentrated antibody solution contains 330 mg/ml or more of antibody/antibody fragments.
117. A method according to any of the preceding embodiments, wherein the concentrated antibody solution contains 335 mg/ml or more of antibody/antibody fragments.
118. A method according to any of the preceding embodiments, wherein the concentrated antibody solution contains 340 mg/ml or more of antibody/antibody fragments.
119. A method according to any of the preceding embodiments, wherein the concentrated antibody solution contains 345 mg/ml or more of antibody/antibody fragments.
120. A method according to any of the preceding embodiments, wherein the concentrated antibody solution contains 350 mg/ml or more of antibody/antibody fragments.
121. A method according to any of the preceding embodiments, wherein the solution is heated to a temperature of at least 30° C. during filtration.
122. A method according to any of the preceding embodiments, wherein the solution is heated to a temperature of at least 31° C. during filtration.
123. A method according to any of the preceding embodiments, wherein the solution is heated to a temperature of at least 32° C. during filtration.
124. A method according to any of the preceding embodiments, wherein the solution is heated to a temperature of at least 33° C. during filtration.
125. A method according to any of the preceding embodiments, wherein the solution is heated to a temperature of at least 34° C. during filtration.
126. A method according to any of the preceding embodiments, wherein the solution is heated to a temperature of at least 35° C. during filtration.
127. A method according to any of the preceding embodiments, wherein the solution is heated to a temperature of at least 36° C. during filtration.
128. A method according to any of the preceding embodiments, wherein the solution is heated to a temperature of at least 37° C. during filtration.
129. A method according to any of the preceding embodiments, wherein the solution is heated to a temperature of at least 38° C. during filtration.
130. A method according to any of the preceding embodiments, wherein the solution is heated to a temperature of at least 39° C. during filtration.
131. A method according to any of the preceding embodiments, wherein the solution is heated to a temperature of at least 40° C. during filtration.
132. A method according to any of the preceding embodiments, wherein the solution is heated to a temperature of at least 41° C. during filtration.
133. A method according to any of the preceding embodiments, wherein the solution is heated to a temperature of at least 42° C. during filtration.
134. A method according to any of the preceding embodiments, wherein the solution is heated to a temperature of at least 43° C. during filtration.
135. A method according to any of the preceding embodiments, wherein the solution is heated to a temperature of at least 44° C. during filtration.
136. A method according to any of the preceding embodiments, wherein the solution is heated to a temperature of at least 45° C. during filtration.
137. A method according to any of the preceding embodiments, wherein the solution is heated to a temperature of at least 46° C. during filtration.
138. A method according to any of the preceding embodiments, wherein the solution is heated to a temperature of at least 47° C. during filtration.
139. A method according to any of the preceding embodiments, wherein the solution is heated to a temperature of at least 48° C. during filtration.
140. A method according to any of the preceding embodiments, wherein the solution is heated to a temperature of at least 49° C. during filtration.
141. A method according to any of the preceding embodiments, wherein the solution is heated to a temperature of at least 50° C. during filtration.
142. A method according to any of the preceding embodiments, wherein the solution is heated to a temperature of at least 51° C. during filtration.
143. A method according to any of the preceding embodiments, wherein the solution is heated to a temperature of at least 52° C. during filtration.
144. A method according to any of the preceding embodiments, wherein the solution is heated to a temperature of at least 53° C. during filtration.
145. A method according to any of the preceding embodiments, wherein the solution is heated to a temperature of at least 54° C. during filtration.
146. A method according to any of the preceding embodiments, wherein the solution is heated to a temperature of at least 55° C. during filtration.
147. A method according to any of the preceding embodiments, wherein the solution is heated to a temperature of at least 56° C. during filtration.
148. A method according to any of the preceding embodiments, wherein the solution is heated to a temperature of at least 57° C. during filtration.
149. A method according to any of the preceding embodiments, wherein the solution is heated to a temperature of at least 58° C. during filtration.
150. A method according to any of the preceding embodiments, wherein the solution is heated to a temperature of at least 59° C. during filtration.
151. A method according to any of the preceding embodiments, wherein the solution is heated to a temperature of at least 60° C. during filtration.
152. A method according to any of the preceding embodiments, wherein the solution is heated to a temperature of at least 61° C. during filtration.
153. A method according to any of the preceding embodiments, wherein the solution is heated to a temperature of at least 62° C. during filtration.
154. A method according to any of the preceding embodiments, wherein the solution is heated to a temperature of at least 63° C. during filtration.
155. A method according to any of the preceding embodiments, wherein the solution is heated to a temperature of at least 64° C. during filtration.
156. A method according to any of the preceding embodiments, wherein the solution is heated to a temperature of at least 65° C. during filtration.
157. A method according to any of the preceding embodiments, wherein the solution is heated to a temperature of at least 66° C. during filtration.
158. A method according to any of the preceding embodiments, wherein the solution is heated to a temperature of at least 67° C. during filtration.
159. A method according to any of the preceding embodiments, wherein the solution is heated to a temperature of at least 68° C. during filtration.
160. A method according to any of the preceding embodiments, wherein the solution is heated to a temperature of at least 69° C. during filtration.
161. A method according to any of the preceding embodiments, wherein the solution is heated to a temperature of at least 70° C. during filtration.
162.
163. A method according to any of the preceding embodiments, wherein the solution is heated to a temperature of at least 30° C. during filtration.
164. A method according to any of the preceding embodiments, wherein the solution is heated to a temperature of at least 30° C. during filtration.
165. A method according to any of the preceding embodiments, wherein the solution is heated to a temperature of at least 30° C. during filtration.
166. A method of filtration according to any of the preceding embodiments, wherein the flow through the filter is constant during the filtration.
167. A method of filtration according to any of preceding embodiments, wherein the flow rate through the filter is increased compared to filtration performed at a room temperature.
168. A method according to any preceding embodiments, wherein the filter flow rate is increased by a factor of at least 10.
169. A method according to any preceding embodiments, wherein the filter flow rate is increased by a factor of at least 15.
170. A method according to any preceding embodiments, wherein the filter flow rate is increased by a factor of at least 20.
171. A method according to any preceding embodiments, wherein the filter flow rate is increased by a factor of at least 25.
172. A method according to any of embodiments 1-167, wherein the filter flow rate is constant and more than 100% increased compared to a filtration performed at room temperature.
173. A method of filtration according to any of the preceding embodiments, wherein the speed of filtration is increased compared to a filtration performed at a room temperature.
174. A method of filtration according to any of the preceding embodiments, wherein protein/antibody content in filtrate is same or increased, as compared to a filtration at a room temperature.
175. A method of filtration according to any of the preceding embodiments, wherein there is no increase in %HMWP compared to a filtration at a room temperature.
176.
177. A method of dead end filtration of a highly concentrated solution containing an antibody and/or a fragment thereof, with a membrane filtration medium, wherein the solution is heated to a temperature ranging from 30-70° C. during and/or before filtration.
178. A method of increasing the filtration yield during a dead end filtration of a highly concentrated solution containing an antibody and/or a fragment thereof, wherein the solution is heated to a temperature ranging from 30-70° C. during and/or before filtration.
179. A method according to embodiment 177, wherein the solution is heated to a temperature of at least 40° C. during and/or before filtration.
180. A method according to embodiment 177, wherein the solution is heated to a temperature of at least 45° C. during and/or before filtration.
181. A method according to embodiment 177, wherein the solution is heated to a temperature between 40° C. and 50° C. during and/or before filtration.
182.
183. A method of increasing the filtration flow rate during a dead end filtration of a highly concentrated solution containing an antibody and/or a fragment thereof, wherein the solution is heated to a temperature ranging from 30-70° C. during and/or before filtration.
184. A method of keeping the filtration flow rate constant during a dead end filtration of a highly concentrated solution containing an antibody and/or a fragment thereof, wherein the solution is heated to a temperature ranging from 30-70° C. during and/or before filtration.
185. A method of stabilising the flow rate during a dead end filtration of a highly concentrated solution containing an antibody and/or a fragment thereof, wherein the solution is heated to a temperature ranging from 30-70° C. during and/or before filtration.
186. A method of stabilising and increasing the flow rate during a dead end filtration of a highly concentrated solution containing an antibody and/or a fragment thereof, wherein the solution is heated to a temperature ranging from 30-70° C. during and/or before filtration.
187. A method according to any of embodiments 181-184, wherein the solution is heated to a temperature of at least 40° C. during and/or before filtration.
188. A method according to any of embodiments 181-184, wherein the solution is heated to a temperature of at least 45° C. during and/or before filtration.
189. A method according to any of embodiments 181-184, wherein the solution is heated to a temperature between 40° C. and 50° C. during and/or before filtration.
190. A method of according to any of embodiments 181-187, wherein the flow rate through the filter is increased compared to filtration performed at a room temperature.
191. A method according to any of embodiments 181-188, wherein the filter flow rate is increased by a factor of at least 10.
192. A method according to any any of embodiments 181-189, wherein the filter flow rate is increased by a factor of at least 15.
193. A method according to any of embodiments 181-190, wherein the filter flow rate is increased by a factor of at least 20.
194. A method according to any of embodiments 181-191, wherein the filter flow rate is increased by a factor of at least 25.
195. A method according to any of embodiments any of embodiments 181-188, wherein the filter flow rate is constant and more than 100% increased compared to a filtration performed at room temperature.
196. A method according to any of embodiments 181-193, wherein the concentrated antibody solution to be filtrated contains at least 100 mg/ml of antibody/antibody fragments.
197. A method according to any of embodiments 181-194, wherein the concentrated antibody solution to be filtrated contains at least 110 mg/ml of antibody/antibody fragments.
198. A method according to any of embodiments 181-195, wherein the concentrated antibody solution to be filtrated contains at least 125 mg/ml of antibody/antibody fragments.
199. A method according to any of embodiments 181-196, wherein the concentrated antibody solution to be filtrated contains at least 150 mg/ml of antibody/antibody fragments.
200. A method according to any of embodiments 181-194, wherein the concentrated antibody solution to be filtrated contains at least 175 mg/ml of antibody/antibody fragments.
201. A method according to any of embodiments 181-194, wherein the concentrated antibody solution to be filtrated contains at least 200 mg/ml of antibody/antibody fragments.
202. A method of filtration according to any of embodiments 181-194 wherein there is no increase in %HMWP compared to a filtration at a room temperature.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of stabilising and/or increasing the flow rate during a dead end filtration of a highly concentrated solution containing an antibody and/or a fragment thereof, wherein the solution is heated to a temperature ranging from 30-70° C. during and/or before filtration.

2. A method of stabilising and/or increasing the flow rate during a dead end filtration according to claim 1, wherein the solution is heated to a temperature of at least 40° C. during and/or before filtration

3. A method of stabilising and/or increasing the flow rate during a dead end filtration according to claim 1, wherein the yield is increased compared to filtration at room temperature.

4. A method of stabilising and/or increasing the flow rate during a dead end filtration according to claim 1, wherein the flow through the filter is constant during the filtration.

5. A method of stabilising and/or increasing the flow rate during a dead end filtration according to claim 1, wherein the filter capacity is constant through the filtration.

6. A method of stabilising and/or increasing the flow rate during a dead end filtration according to claim 1, wherein the flow rate through the filter is increased compared to filtration at a room temperature.

7. A method of stabilising and/or increasing the flow rate during a dead end filtration according to claim 1, wherein the concentration of the antibody or fragment thereof in the concentrated solution is above 200 g/L.

8. A method of stabilising and/or increasing the flow rate during a dead end filtration according to claim 1, wherein the solution is heated to a temperature of at least 45° C. during and/or before filtration

9. A method of stabilising and/or increasing the flow rate during a dead end filtration according to claim 1, wherein the solution is heated to a temperature of 40° C. during and/or before filtration.

10. A method of stabilising and/or increasing the flow rate during a dead end filtration according to claim 1, wherein the solution is heated to a temperature of 45° C. during and/or before filtration

11. A method of stabilising and/or increasing the flow rate during a dead end filtration according to claim 1, wherein the antibody is a monoclonal antibody.

12. A method of stabilising and/or increasing the flow rate during a dead end filtration according to claim 1, wherein the antibody is an anti-IL-20 monoclonal antibody.

13. A method of stabilising and/or increasing the flow rate during a dead end filtration according to claim 1, wherein the antibody is an anti-TFPI monoclonal antibody.
